(19) United States
(12) Patent Application Publication
(10) Pub. No.: US 2009/0130018 A1
(43) Pub. Date: May 21, 2009

(54) SUPRAMOLECULAR AGGREGATES CONTAINING CHELATING AGENTS AND BIOACTIVE PEPTIDES AS EFFECTIVE AND SELECTIVE DELIVERY TOOLS FOR DRUGS AND CONTRAST AGENTS IN MRI OR NUCLEAR MEDICINE

(76) Inventor: Antonella ACCARDO, Torre del Greco (IT)

Correspondence Address: Steinfl & Bruno, 301 N Lake Ave Ste 810, Pasadena, CA, 91101, US

(21) Appl. No.: 11/915,882

(22) PCT Filed: May 26, 2006
(86) PCT No.: PCT/EP06/05046

§ 371 Date: Jan. 25, 2008

Related U.S. Application Data
(60) Provisional application No. 60/685,538, filed on May 31, 2005.

(51) Int. Cl. A61K 51/12; A61K 49/14

(52) U.S. Cl. 424/1.21; 424/9.34

(57) ABSTRACT

The invention relates to supramolecular aggregates obtained by coaggregation of two monomers: —a first monomer containing a paramagnetic or radioactive metal ion complexed by a chelating agent having a lipophilic moiety, —a second monomer containing a bioactive peptide linked to a lipophilic moiety through an organic spacer. The aggregates are selectively driven by the exposed bioactive peptide on a desired biological target. The aggregates (micelles, vesicles or liposomes) could entrap on their inner region or on their surface a pharmaceutical active principle (drug). The invention thus relates to target specific delivery of drugs and/or metal ions. The invention therefore provides compositions containing said aggregates for use as contrast agents, in nuclear medicine or MRI, for the selective delivery of drugs, and for simultaneous delivery of a drug and a metal ion.

The invention relates to supramolecular aggregation of two monomers containing a chelating agent able to coordinate a paramagnetic (Gadolinium) or radioactive metal ion (Indium, Lutetium, Yttrium, Gallium) having a lipophilic moiety and a second monomer containing a bioactive peptide linked to a lipophilic moiety. The aggregates are selectively driven by the exposed bioactive peptide on the desired biological target. The aggregates (micelles, vesicles or liposomes) can entrap a pharmaceutical active principle (drug) in their inner compartment or in the phospholipid bilayer. The invention thus relates to specific delivery of drugs and/or metal ions.

The aggregates of this invention are useful as: i) selective vehicles of contrast agents for both Magnetic Resonance Imaging and Nuclear Medicine techniques; ii) selective vehicles for simultaneous delivery of a drug (in the inner compartment) and a contrast agent (on the aggregate surface, for its visualization); iii) selective delivery of a drug and a therapeutic active radionuclide. The invention also concerns the preparation of the compositions, as well as injectable aggregates, their use and a kit comprising dry aggregates and a physiologically acceptable aqueous carrier.

### BACKGROUND OF THE INVENTION

Clinical diagnostic imaging requires emissions of signals from pathological areas of interest to achieve differentiate structures from surrounding tissues. Current imaging modalities include gamma-scintigraphy (in nuclear medicine based on the application of γ emitting radioactive nucleus) and magnetic resonance (MR, based on the relaxation rate of proton of water molecule by enhancing with paramagnetic ions). The critical point for these techniques is that both require contrast agents able to recognise the tumours cells. In recent years, specific tools were developed to target tumours cells based on bioactive molecules. In the last ten years five somatostatin receptor sub-classes (see e.g. U.S. Pat. No. 5,436,155; WO 97/14715; Biochemical and Biophysical Research Communications 258, 689-694, 1999) and two cholecystokinin type A and B (see e.g. WO98/51337 and WO98/35707) receptors have been evidenced, all belonging to the family of G-proteins associated receptors, overexpressed in many different cancers. Many peptide conjugates able to bind these receptors with high affinity are synthesized and used in nuclear medicine applications (J. C. Reubi, Cancer Research 57, 1377, 1997, and WO9731657, J. Nucl. Med. 41, 1704-1713, 2000; Current Medicinal Chemistry 7, 971-994, 2000) and some of them are commercially available (see e.g. Octreoscan®).

The quantity of the contrast agents to be accumulated in the area of interest is quite different in the two techniques. The very sensitive nuclear medicine needs very low concentration (10⁻¹⁰ M) of radionuclide to give images, while MRI gives very resolved images only when the concentration is higher than 10⁻⁴M of contrast agent. Various attempts to produce substance suitable to increase the relaxivity were performed. Two approaches were followed to improve this parameter: delaying the reorientation time by increasing the molecular size of the chelating agents and increasing the number of ions delivered to the target. The two goals can be achieved developing carriers for the chelating agents such as linear polymers, dendrimers, liposomes, micelles and other microparticulates. In the first two cases, the chelating agents are covalently bound to the macromolecules. Brechbiel developed large dendrimer macromolecular MRI agents with diamine core bearing a large number of chelating groups. Following this approach in last years lysine dendrimers functionalized with bioactive peptides were synthesised to target tumours cells (WO 03/014157). Aime et al (Chem. Eur. J., 6, 2609-2617, 2000) formulated lysines or ornithines cationic linear polymer able to interact with new anionic chelating groups. In order to increase the number of contrast agents the most promising compounds are supramolecular aggregates. Recently, (U.S. Pat. No. 5,833,948) supramolecular mixed associations containing chelating group able to coordinate paramagnetic metals bound to lipophilic moiety and non-ionic surfactant were formulated. These aggregates show high relaxivity value and are particularly suitable for use in NMR blood pool imaging of organs in human or animal body.

Wu (US 200302290017) developed a solid phase synthesis method to preparing peptide spacer lipid conjugates in order to formulate liposome bearing bioactive peptide. Liposomes are able to carry encapsulated molecules selectively in tumour cells. These aggregates can delivery on the target drugs or contrast agents contained in liposome. These aggregates are able to bind receptor overexpressed with high affinity but have less opportunity to be recognized by the reticuloendothelial system.

The use of aggregates (micelles, vesicles or liposomes) as carriers of drugs or contrast agents has been validated in the last few years. The most relevant systems are described by V. P. Torchilin (Nature Reviews, Drug Discovery, Vol. 4, 145, 2005). Anyway, all the reported aggregates have been developed as delivery systems for a drug or for a contrast agent. None of them are capable of the simultaneous delivery of a drug (in the inner compartment) and a contrast agent (on the aggregate's surface, for their visualization); or of the selective delivery of a drug and a therapeutic radionuclide, such as a beta-emitting metal ion.

### SUMMARY OF THE INVENTION

The invention relates to supramolecular aggregates obtained by coaggregation of two monomers:
a first monomer containing a paramagnetic or radioactive metal ion complexed by a chelating agent having a lipophilic moiety,
a second monomer containing a bioactive peptide linked to a lipophilic moiety through an organic spacer.

The aggregates are selectively driven by the exposed bioactive peptide on a desired biological target. The aggregates (micelles, vesicles or liposomes) could entrap on their inner region or on their surface a pharmaceutical active principle (drug). The invention thus relates to target specific delivery of drugs and/or metal ions. The aggregates object of this invention could act as: i) selective vehicles of contrast agents for both Magnetic Resonance Imaging and Nuclear Medicine techniques; ii) selective vehicles for simultaneous delivery of a drug (in the inner compartment) and a contrast agent (on the aggregate surface, for its visualization); iii) selective delivery of a drug and a therapeutic active radionuclide such as a beta-emitting metal ion.

The invention therefore provides compositions containing said aggregates for use as contrast agents, in nuclear medicine or MRI, for the selective delivery of drugs, and for simultaneous delivery of a drug and a metal ion.

### DESCRIPTION OF THE INVENTION

The monomer containing the chelating agent comprises a polyaminopolycarboxylate moiety, able to give stable complexes with the metal ion of interest such as Gd(III), In(III), Y(III), Ga(III) and Lu(III) covalently bound to an organic compound carrying at least two lipophilic substituents such as ester of a fatty alcohol.

The monomer containing the bioactive peptide contains the bioactive peptide, a spacer of suitable length to leave the peptide far away from the hydrophobic moiety, and an organic compound carrying at least two lipophilic substituents such as ester of a fatty alcohol, identical or similar to that present on the other monomer.

The two monomers can also be linked together with a covalent bond, in this case each monomer could contain only one lipophilic substituent on the organic moiety. The covalent binding between the monomer could be obtained by UV radiation induced reaction on C—C unsatured bonds present on the lipophilic tails.

The two monomers can also be substituted by a single monomer bearing in the same molecule the two lipophilic tails, the chelating agent and the bioactive peptide.

Optionally, the composition of the two monomers may also include one or more amphipatic compounds e.g. phospholipids or non ionic surfactants.

The supramolecular adduct, consisting of the two monomers and optionally other amphipatic compounds, will be assembled in a mixed micelle with spherical or rodlike shape, or in a vesicle or in a double strand, or in liposomes. In all the possible structures, the hydrophilic components (metal complex and bioactive peptide) will be present on the aggregate surface. In all the possible structures a drug can be entrapped in the inner compartment or in the phospholipid bilayer region.

The ratio between the monomer containing the metal complex and the monomer containing the bioactive peptide can vary between 10 and 0.5, preferably between 5 and 2.

The size of the supramolecular aggregates will depend on the monomers used in the formulation and on the preparation method of the aggregates: the size of the micelles usually ranges between 5 and 50 nm, the size of vesicles and liposomes ranges between 50 and 500 nm; thickness of lipid bilayers, double strand aggregates ranges between 5-20 nm.

The peptide moiety will be completely exposed on the surface of the supramolecular adduct and will interact with its specific membrane receptor. In this way, the entire supramolecular adduct will interact selectively with cells expressing the membrane receptor for the desired peptide.

Moreover, due to entrapment in the supramolecular adduct, the Gadolinium complex will exhibit higher relaxivity than simple Gd complexes.

The high relaxivity of each Gd complex, the high number of Gd complexes for each supramolecular adduct and the target selectivity due to exposed bioactive peptide, will allow to the supramolecular adducts of this invention to act as specific and selective MRI contrast agents.

Moreover, the adduct may also contain other metal ions, such as ¹¹¹In(III), ⁹⁰Y(III), ⁶⁸Ga(III) ⁶⁷Ga(III) or ¹⁷⁷Lu(III), complexed by the monomer containing the chelating moiety; in this case the adduct can be used as selective and specific contrast agent in Nuclear Medicine.

The supramolecular aggregates may entrap a pharmaceutical active ingredient in the inner compartment or in the phospholipid bilayer region, for the selective target of cancerous cells. A metal ion may be used as radioactive marker for nuclear medicine techniques thus allowing the visualization of the biodistribution behaviour of the drug containing aggregates. Alternatively, the metal ion such as a beta-emitting radionuclide, may be used as radio-therapeutic agent, thus enhancing the therapeutic activity of the contained drug.

### DETAILED DESCRIPTION OF THE INVENTION

It has been found that selective and specific contrast agents or drug delivery systems or a system containing simultaneously a drug and a metal ion, may be obtained by assembling together in a supramolecular adduct two different monomeric components, one containing a bioactive peptide able to give high selectivity and specificity to the entire adduct and the other containing a stable complex of metal ions such gadolinium(III) or Indium(III) acting as very effective contrast agents in MRI or NM. Both monomers contain a lipophilic moiety consisting of two hydrophobic tails allowing the formation of the supramolecular adducts. The two monomers can also be substituted by a single monomer bearing in the same molecule the two lipophilic tails, the chelating agent and the bioactive peptide.

The aggregates (micelles, vesicles or liposomes) of the invention are able to encapsulate pharmaceutical active ingredients in their inner compartment or in their phospholipid bilayer region.

The present invention is characterised by the presence of the chelating agent allowing the aggregates to achieve several goals according to the metal ion complexed:
(a) A high relaxivity and target selective contrast agent for MRI application if the metal is a paramagnetic ion (Gd(III), Mn(II), Cr(III), Cu(II), Fe(III), Pr(III), Nd(III) Sm(III), Tb(III), Yt(III) Dy(III), Ho(III) and Er(III)).
(b) Exceptionally effective and selective drug delivery systems containing a marker able to monitor the circulation and the biodistribution of the drug containing aggregate in vivo by using the Nuclear Medicine techniques if the metal is γ-emitting (¹¹¹In(III), ^{99m}Tc(V), ⁶⁷Ga(III)) isotope.
(c) Exceptionally effective and selective drug delivery systems and powerful radiotherapeutic compounds containing, simultaneously, a drug entrapped in the inner compartment of the aggregate, or on its phospholipidic bilayer region, and a beta-emitting radioactive metal ion (⁹⁰Y(III) ¹⁷⁷Lu(III)) complexed by the chelating agent.

Exceptionally effective and selective MRI contrast agents can be obtained in presence of:
1—bioactive peptide on the external surface of the supramolecular adduct, carrying the supramolecular adduct to the desired target in a selective and specific way;
2—high number of paramagnetic ions (i.e.; Gadolinium) complexed to the chelating agents allocated on the supramolecular adduct by a covalent bond to lipophilic moiety.

Exceptionally effective and selective drug delivery systems can be obtained in presence of:
1. bioactive peptide on the external surface of the supramolecular adduct, driving the supramolecular adduct to the desired target in a selective and specific way;
2. a pharmaceutical active ingredient (drug) entrapped in the inner compartment of the aggregate or embedded in the aggregate phospholipid bilayer region;
3. a radioactive γ-emitting metal ion as a marker to monitor, by nuclear medicine techniques, the biodistribution properties of the aggregates and of the contained drug.

Exceptionally effective and selective drug delivery systems and powerful radiotherapeutic compounds containing, simultaneously, a drug entrapped in the inner compartment of the aggregate or on its phospholipidic bilayer region and a beta-emitting radioactive metal ion (⁹⁰Y(III) ¹⁷⁷Lu(III)) complexed by the chelating agent; the beta-emitting radionuclide enhancing the therapeutic activity of the contained drug.
1. bioactive peptide on the external surface of the supramolecular adduct therefore able to drive the supramolecular adduct to the chosen target in a selective and specific way;
2. a pharmaceutical active principle (drug) entrapped in the inner compartment of the aggregate or embedded in the aggregate phospholipid bilayer region;
3. a β-emitting radionuclide, as radio-therapeutic agent, able to reinforce the therapeutic activity of the contained drug.

As schematically shown in FIG. 1, the monomers that aggregate in a supramolecular adduct should contain few functional units.

In particular, the monomer containing the chelating agent should comprise the organic moiety with the two hydrophobic tails, and a bifunctional chelate able to bind the metal of interest with high stability. The description of the single units is reported below.
The first unit, common to both monomers, consists of a hydrophobic moiety, containing two hydrocarbon tails, which readily couple or intertwine (presumably by Van der Waals forces) with the hydrophobic part of the other monomers. Two hydrocarbon tails should be present for each hydrophobic moiety; in fact the presence of two tails allows the formation of different structured supramolecular aggregates: micelles (in which the hydrophobic moieties collapse together in the micelle hydrophobic core) or vesicles, liposomes and lipidic bilayer (in which the two hydrocarbon tails of each monomer interact to form double strands of hydrophobic regions, leaving the covalently bound hydrophobic parts of the monomers exposed to the solution). Moreover, monomers containing only one hydrocarbon tail have an haemolytic effects and the supramolecular aggregates formed by aggregation of monomers bearing single-tail hydrophobic moieties result toxic in vivo, while monomers bearing two hydrophobic tails, do not present haemolytic effects and toxicity problems due to their higher similarity with phospholipids, the main component of biological membranes (Rif. Anelli, P. L.; Magnetic Resonance Materials in Physics, Biology and Medicine, (2001), 12, 114-120).

The hydrophobic moiety could be obtained, for instance, in the form of carboxylate ester with hydrophobic aliphatic or aromatic alcohols. Said alcohols include saturated and unsaturated C8 to C24 alcohols like methanol, ethanol, propanol, butanol (n-, iso-, tert-), pentanol, hexanol (and isomers), heptanol, octanol (and isomers), nonanol, decanol and fatty alcohols; suitable aromatic alcohols are optionally substituted benzyl- and higher phenylalkyl-alcohols. The hydrophobic moiety could be also obtained, for instance, in the form of carboxylate amide with hydrophobic aliphatic or aromatic amines. Said amines may be saturated and unsaturated C8 to C24 amines like methylamine, ethylamine, propylamine, butylamine (n-, iso-, tert-), pentylamine, hexylamine (and isomers), octylamine (and isomers), nonylamine, decylamine, aminoadamantane and fatty amines; aromatic amines include substituted and unsubstituted benzyl- and higher phenylalkyl-amines. The most effective results are obtained when the number of carbon atoms in the hydrocarbon tail is comprised between 12 and 18. The aggregates obtained seem reasonably stable even with short alkyl groups; however, for merely practical reasons, C12-C18 alkyl groups are preferred. In fact, aggregates having longer alkyl chains have lower critical micelle concentration (cmc) values. The hydrocarbon chain in the monomers can be saturated, unsaturated or polyunsaturated.
The second unit present in this monomer is a chelating agent yielding stable complexes with the metal ion of interest. Polyaminopolycarboxylic acids are particularly useful for complexing the paramagnetic ions intended for NMR imaging or radioactive isotopes of metal ions intended for imaging by Nuclear Medicine techniques. The chelating moiety may be selected from EDTA, DTPA, DTPAGlu, DTPALys, DTPASer, BOPTA, DOTA, DO3A and/or their derivatives, all containing a free function unit for covalent linkage to the other monomer units.
The third unit present on this monomer is the metal ion of interest: a paramagnetic metal for MRI application or a radioactive isotope of a metal ion intended for imaging or therapy by Nuclear Medicine techniques. The paramagnetic metal may be selected from well known paramagnetic metals, notably from Gd(III), Mn(II), Cr(III), Cu(II), Fe(III), Pr(III), Nd(III) Sm(III), Tb(III), Yt(III) Dy(III), Ho(III) and Er(III). The radioactive metal may be selected from well known group of ¹¹¹In(III), ^{99m} Tc(V), ⁹⁰Y(III) ¹⁷⁷Lu(III) ⁶⁸Ga(III).

According to the invention, a spacer may be introduced between the chelating agent and the lipophilic chains. The spacer is a linear hydrophilic polymer chain containing a linkage functional group at each end of the chain for attaching the chelating agent and the lipophilic chains.

The monomer containing the peptide should consists of the organic moiety with the two hydrophobic tails, a spacer separating the bioactive peptide from the surface of the aggregate and by the bioactive peptides. The single units are disclosed below.
The first unit is similar or the same as that on the other monomer: it consists of a hydrophobic moiety, containing two hydrocarbon tails, which readily couples or intertwines (presumably by Van der Waals forces) with the hydrophobic part of the other monomers. Two hydrocarbon tails should be present for each hydrophobic moiety, the presence of two tails allowing the formation of different structured supramolecular aggregates: micelles (in which the hydrophobic moieties collapse together in the micelle hydrophobic core) or vesicles, liposomes and lipidic bilayer (in which the two hydrocarbon tails of each monomer interact to form double strands of hydrophobic regions, leaving the covalently bound hydrophobic parts of the monomers exposed to the solution). Moreover, monomers containing only one hydrocarbon tail have a haemolytic effects and the supramolecular aggregates formed by aggregation of monomers bearing single-tail hydrophobic moieties result toxic in vivo, while monomers bearing two hydrophobic tails, do not present haemolytic effects and toxicity problems due to their higher similarity with phospholipids, the main component of biological membranes.

The hydrophobic moiety could be obtained, for instance, in the form of carboxylate ester with hydrophobic aliphatic or aromatic alcohols. Suitable alcohols comprise saturated or unsaturated C8 to C24 alcohols such as methanol, ethanol, propanol, butanol (n-, iso-, tert-), pentanol, hexanol (and isomers), heptanol, octanol (and isomers), nonanol, decanol and fatty alcohols; suitable aromatic alcohols comprise substituted and unsubstituted benzyl- and higher phenylalkyl-alcohols. The hydrophobic moiety could be also obtained, for instance, in the form of carboxylate amide with hydrophobic aliphatic or aromatic amines. Said amines may be saturated and unsaturated C8 to C24 amines like methylamine, ethylamine, propylamine, butylamine (n-, iso-, tert-), pentylamine, hexylamine (and isomers), octylamine (and isomers), nonylamine, decylamine, aminoadamantan and fatty amines; suitable aromatic amines are optionally substituted benzyl- and higher phenylalkyl-amines. The most effective results are obtained when the number of carbon atoms in the hydrocarbon tail ranges between 12 and 18. The aggregates obtained seem reasonably stable even with short alkyl groups however, for merely practical reasons, C12-C18 alkyl groups are preferred. In fact, aggregates having longer alkyl chains have lower critical micelle concentration (cmc) values. The hydrocarbon chain in the monomers can be saturated, unsaturated or polyunsaturated.
The second unit in this monomer is a spacer. The presence of the spacer is mandatory to maintain the bioactive peptide far away from the hydrophobic core and exposed out from the supramolecular aggregate. The spacer should be therefore introduced between the peptide and the lipophilic chains. The spacer is a linear hydrophilic polymer chain containing a linkage functional group at each end of the chain for attaching the peptide and the lipophilic chains. The suitable spacers in the present invention include for example polyglycine, polyethylene glycol, polypropylene glycol, polymethacrylamide, polyhydroxyethylacrylate, polyhydroxypropylmethacrylate, 8-amino-3,6-dioxaoctanoic acid and polyoxyalkene. The preferred spacers are polyethylene glycols, having a molecular weight between 100-10000 daltons, more preferably between 100-5000 daltons.
The third unit in this monomer is the bioactive peptide driving the entire supramolecular aggregate to the biological target. Several types of molecules, such as antibodies, proteins, small synthetic molecules and peptides, have been developed as the targeting ligands for binding the target sites. Peptides are considered as highly effective targeting ligands, since a peptide can serve as a recognition component in protein-protein interactions such as receptor-ligand interactions.

Preferred bioactive branched or linear peptides for use according to the invention have the following sequence of general formula (I) or (II)

(AA₀)_{w}-AA₁-AA₂-AA₃-Gly-Trp-AA₆-Asp-PheR₂ (I)

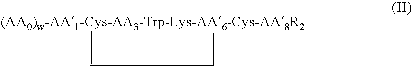

wherein

AA₀ AdOO or other spacer

AA₁ Asp or Glu

AA₂ Tyr or SO₃H-Tyr

AA₃ Met or Nle or Leu

AA₆ Met, Nle or Leu

AA′₁, AA′₃, AA′₆, and AA′8 are any amino acid, either natural or not, in L or D configuration; or

AA′₈, is an amino alcohol derivative from any amino acid, either natural or not, in L or D configuration.

Furthermore, many cellular membrane receptors associated with specific diseases have been studied. Peptides, such as CCK-peptides, RGD-peptides, somatostatin, cortistatin, octreotide, bombesin, chemotactic peptides, vasoactive intestinal peptide, Her2 ligands, NPY analogs, CXCR4 ligands such as the T140 peptide, Integrin binding ligands, fibroblast growth factor, hepatocyte growth factor, epidermal growth factor, laminin binding ligands, nerve growth factor, fibronectin, fibroblast growth factor, insulin like growth factor, vascular endothelial growth factor receptor ligands, a platelet derived growth factor receptor ligands and transforming growth factor receptor ligands and mimetics thereof or ligands for other plasma membrane receptor or other cell surface component of cancer cells, are good candidates as targeting ligands. Many receptors of these peptides have been found being overexpressed in various tumor cells. Moreover, peptides and peptide mimetics have several unique advantages over other types of molecules (e.g., antibodies). Generally, these peptides bind to target cells with a ligand-receptor association with high affinity and enter the intercellular compartments through receptor-mediated endocytosis. However, an antibody-based targeted supramolecular aggregate may not utilize the endocytosis pathway into the interior of the cells by the antigen on the cell membrane. Furthermore, peptides have less opportunity to be recognized by the reticuloendothelial system and are thus cleared from the blood circulation system. Peptide mimetics can provide a higher binding affinity and a better resistance to the proteases degradation than natural peptides. According to the invention, the peptide is covalently linked to the spacer so as to maintain its biological activity as well as the receptor recognition and binding.

The peptide containing monomer may also consist of the bioactive peptide covalently bound, through the spacer, to a phospholipid moiety. The mPEG-DSPE is a suitable unit comprising in the same molecule the phospholipid moiety and the PEG spacer. This kind of monomer is the preferred component to obtain functionalised mixed liposomes.

The two monomers can be substituted by a single monomer bearing in the same molecule the hydrophobic unit having the two lipophilic tails, the chelating agent, a spacer and the bioactive peptide (see FIGS. 2.a and 2.b).

The two single monomers can also be substituted by Gemini (dimeric) compounds in which two identical monomers are connected at the level of, or close to, the head groups by a spacer group. In this case, the single monomer can contain one or two hydrophobic tails. The two monomers can be linked together with a covalent bond such as an amide bond, an ester or thioester bond or a disulphide bond (see FIGS. 3.a and 3.b). An alkyl chain or an aromatic moiety can be used as symmetric or asymmetric spacer between the two monomers.

The two monomers can also be linked together with a covalent bond obtained by UV-induced reaction on C—C unsatured bonds present on the lipophilic tails; in this case, each monomer may contain only one lipophilic substituent on the organic moiety.

The supramolecular aggregates are prepared according to different procedures.

In the first preparative mode (mode 1), employed to obtain mixed micelles, the monomer solutions are stirred at room temperature until complete dissolution and then used without further treatment. The ratio between the solutes can be selected so as to have an average of 3-10 peptide derivatives per micelle.

In a second preparative mode (mode 2), employed to obtain mixed vesicles or liposomes or double strands, a monomers combination is dissolved in a suitable organic solvent such as chloroform, methylene chloride, methanol, or mixtures thereof. The solution is evaporated to remove any organic solvent, and then dried in vacuum to obtain a lipid film. Moreover, the lipid film is hydrated in an aqueous solution by vortexing. The mixture is finely dispersed in water (or other physiologically liquid carrier), for instance by sonication for about 15 min. Finally, the aqueous supramolecular aggregates are downsized by extrusion technique. The mixture is extruded through double-stacked polycarbonate membranes (pore size from 400 to 100 nm). The aggregate size is determined by Cryo-TEM technique.

After the preparation of vesicles or liposomes by using mode 2, the aggregates may also be stabilized performing photo-activated polymerisation reactions between unsatured bonds present on the hydrocarbon tails of each monomer.

The supramolecular aggregates according to the invention may also contain additional components such as non-ionic, ionic and mixtures of ionic and non-ionic surfactants. Due to their physiological suitability, the non-ionic surfactants are preferred. The non-ionic surfactants are preferably block-copolymers having polyoxyethylene and polyoxypropylene segments, polyethylene glycol-alkyl ethers such as for example polyethylene glycol-octadecyl ether, polyoxyethylene fatty acid esters, polyoxyethylene sorbitan fatty acid esters, n-alkyl glycopyranoside and n-alkyl maltotrioside. The non-ionic surfactant is conveniently selected from commercially available products such as Pluronic®, Poloxamer®, Poloxamine®, Synperonic®, BRIJ®, Myrj®, Tween®s (polysorbates) and their mixtures. The weight proportion of the surfactant relative to the amount of the paramagnetic imaging agent is from 1:50 to 50:1, preferably 1:10 to 10:1, most preferably 1:1.

The supramolecular aggregates according to the invention may also contain additional components such as amphipatic compounds, suitably selected from phospholipids such as 1,2-Dioleoyl-sn-Glycero-3-Phosphocholine (DOPC), 1,2-Dioleoyl-sn-Glycero-3-Phosphoethanolamine (DOPE), 1,2-Distearoyl-sn-Glycero-3-Phosphoethanolamine (DSPE), phosphatidic acid (PA), phosphatidylcholine (PC), phosphatidylethanolamine (PE), phosphatidylserine (PS), phosphatidylglycerol (PG), phosphatidylinositol (PI), cardiolipin (CL) and sphingomyelin (SM). The amphipatic compound may also consists of a mono-phosphate ester of a substituted or partially substituted glycerol, at least one functional group of said glycerol being esterified by saturated or unsaturated aliphatic fatty acid or etherified by saturated or unsaturated alcohol, the other two acidic functions of the phosphoric acid being either free or salified with alkali or earth-alkali metals. Preferably, the phosphate esters will include monophosphates of fatty acid glycerides selected from dimyristoylphosphatidic acid, dipalmitoylphosphatidic acid or distearoylphosphatidic acid.

The phospholipids may also include diacyl and dialkyl glycerophospholipids in which the aliphatic chains have at least 12 carbon atoms, as well as one or more compounds selected from ionic and neutral phospholipids, mono alkyl or alkenyl esters of phosphoric acid and/or cholesterol, ergosterol, phytosterol, sitosterol, lanosterol, and tocopherol. In the compositions containing phospholipids, the weight proportion of the phospholipids to the other monomers seems not critical and it may vary in a wide range e.g. from 1:50 to 50:1. The practical range will be between 10:1 and 1:10, preferably between 1:5 and 5:1, most preferably between 1:3 and 3:1, since the use of a large excess of phospholipid beyond certain concentration does not provide extra benefit.

Upon aggregation, the supramolecular adduct, consisting of one or more monomers and optionally other amphipatic compounds, will be assembled in mixed micelle with spheric or rodlike shape, in vesicles, double strands or in liposomes.

The mixed aggregates as well as the pure monomer aggregates, in the presence or in the absence of the metal, can be characterized for their stability and for their structure. The thermodynamic stability is strictly related to the critical micellar concentration (cmc) which represents the concentration of the monomeric amphiphile at which micelle appears. It can be obtained by surface tension measurements. The lower the cmc value of the amphiphilic molecule, the more stable the micelles are, even at low concentration of an amphiphilic in the medium. This is especially important from the pharmacological point of view, since upon the dilution with a large volume of blood, only aggregates with low cmc value still exist, while aggregates with high cmc value dissociate into monomers.

Structural determinations, such as the aggregation number (N_{agg}), the micellar charge (z), the radius of the aggregates (R), the thickness of the lipidic bilayer in the vesicles or in the liposomes, the height of the double strands, could be performed by FT-PGSE-NMR, fluorescence quenching, small-angle scattering neutron, light scattering and Cryo-TEM physico-chemical techniques.

The supramolecular aggregates can be characterized for their relaxometric properties (at 20 MHz and 25° C. normalised to 1 mM concentration of Gd³⁺ ion) for pure (C18)₂DTPAGlu(Gd) and mixed aggregates (C18)₂DTPAGlu(Gd)—(C18)₂L5CCK8. As it can be seen from this comparative diagram, the contrast agents comprising paramagnetic contrast composition in the form of mixed micelles provides relaxivities which are 30-250% greater than that of the chelating agent-peptide conjugates. Thus, the higher relaxivities coupled to the longer residence times in the circulation obtained with the paramagnetic contrast agents of the invention provide an important advantage in comparison to known NMR contrast agents compositions.

The supramolecular aggregates of the invention can be loaded in the phospholipidic bilayer or the inner cavity with various therapeutic agents. According to the invention, suitable therapeutic agents comprise natural and synthetic antineoplastic compounds. The encapsulation of drugs into liposomes prevents their passive diffusion into non target cells typical of low-molecular-weight agents (typically under 500 g mol⁻¹), thus increasing circulation time in the blood stream and decreasing the accumulation of the drug in non targeted tissues. Moreover, the encapsulated drug is also protected from the often-aggressive in vivo environment.

Loading of the therapeutic agent into liposomes includes the methods of loading water-soluble, hydrophobic and ionic compounds. Water-soluble compounds generally are encapsulated in liposomes by dissolving the agent in an aqueous solution and mixing with a lipid film. Hydrophobic agents can be entrapped into liposomes or incorporated into the lipid bilayer by dissolving the agent with a lipid or lipid combination in a suitable organic solvent and then evaporating the solvent to produce a thin film. Methods for loading ionic agents can be performed by pH, ionic gradient methods as described in Mayer et al., Biochemistry, 27, 2053-2060 (1988) and Haran, G. et al., Biochim. Biophys. Acta., 1151, 201-215 (1993). Examples of these kinds of drugs are doxorubicin, daunorubicin, epirubicin, esorubicin, and idarubicin.

Specific and selective targeting of the mixed supramolecular aggregates can be also characterized in cells expressing the receptor of the bioactive molecule. Experiments performed in control and CCKB receptor overexpressing cells can be utilized to prove receptor interaction of the aggregates both in vitro in cultured cells and in vivo in animal models bearing receptor expressing and control tumors (see the examples 9 and 10 below).

The main aspects of the invention are based on the unexpected finding that exceptionally effective and selective paramagnetic NMR contrast compositions or/and effective and selective drug delivery systems are obtained when two different monomers are used together to obtain supramolecular aggregates. In addition to a metal ion complexed with a polyaminopolycarboxylate chelating agent having a lipophilic moiety, the supramolecular aggregate contains another monomer that provides a bioactive peptide on the external surface of the aggregate capable of driving the supramolecular aggregate to a biological target. Moreover, the entrapment of a drug in the inner compartment of the supramolecular aggregates or in the phospholipid bilayer region allows the preparation of very effective and selective drug delivery systems. The simultaneous presence of a metal ion complexed by the chelating agent allows the in vivo monitoring of the biodistribution behaviour by MRI, if the metal ion is a paramagnetic ion such as Gd(III), or by nuclear medicine techniques, if the metal ion is a gamma or positron emitting nuclide; or it allows to enhance the therapeutic efficacy of the delivered drug if the metal ion is a beta emitting radionuclide.

The configuration of a paramagnetic metal ion bound to an amphipatic structure i.e. a polyaminopolycarboxylate segment comprising a non-ionic hydrophilic function (the spacer) and non-ionic hydrophobic aliphatic chains has shown strikingly high relaxivity values and therefore contrast efficiency in NMR blood pool imaging.

Moreover, the presence in the supramolecular adduct of variable amount of a monomer containing a bioactive peptide allows the supramolecular adduct to interact in a selective way with the target receptor express on cellular membrane.

The ratio between the monomer containing the metal complex and the monomer containing the bioactive peptide may range between 10 and 0.5, preferably between 5 and 2.

The dimension of the supramolecular aggregates will depend on the monomers used in the formulation and on the preparation method of the aggregates: the size of the micelles may range from 5 to 50 nm, the size of vesicles and liposomes may range from 50 to 500 nm; thickness of lipidic bilayers, double strand aggregates, may range from 5 to 20 nm.

The supramolecular aggregates are prepared by simultaneously dispersing the two components of the particulate adduct, for instance the paramagnetic imaging component and the monomer containing the bioactive peptide, in the aqueous carrier liquid at final concentration higher that the relative critical micellar concentration. Optionally, the third component may be added to the dispersion, whereby the addition of said third component will cause the dispersion to become into micellar form.

Once prepared, the dispersion can thereafter be sterilised by heat as usual and used as such, or it can be further dehydrated for storage, for instance by lyophilization. The dehydrated material in form of a powder from which the supramolecular MRI contrast agent or the anticancer drug containing aggregates may be produced by admixing the powder with a portion of carrier liquid and stirring.

Thus, for practically applying the compositions of the invention in the medical field, the dried components and the carrier liquid can be marketed separately in a kit form whereby the contrast agent or the anticancer drug containing aggregates is reconstituted by mixing together the kit components prior to injection.

The first component of the kit, i.e. dry power, may further be stored under a dry inert atmosphere and the second component, a physiologically acceptable carrier liquid, may further contain isotonic additives and other physiologically acceptable ingredients such as mineral salts, vitamins, etc.

As already mentioned, the reconstituted agent is particularly suitable for use in NMR blood pool imaging. These compositions facilitate MR angiography and help to assess myocardial and cerebral ischemia, pulmonary embolism, vascularisation of tumours and tumour perfusion.

The supramolecular adduct can also be used as diagnostics or therapeutics in Nuclear Medicine techniques. According to the invention, the supramolecular aggregates will be formulated using the methods above described, with the chelating monomer in uncomplexed form. Only after supramolecular adduct formulation, the chelating moieties can be labelled with the radioactive metal ion using the known methods.

The invention accordingly provides also pharmaceutical or diagnostic compositions comprising the aggregates of the inventions and suitable excipients. The administration dosages will depend on the active ingredient entrapped into the aggregate, on the kind of condition to be treated or diagnostic procedure to be performed, etc, and will be anyhow determined by the skilled practitioner on the base of the pharmacokinetics, toxicological and pharmacodymanic properties of the selected aggregate.

### DESCRIPTION OF THE DRAWINGS

FIG. 1 is a schematic representation of monomer molecule endowed of two hydrophobic chain: (a) (C18)₂DTPAGlu with the chelating moiety able to conjugate metal ions; (b) (C18)₂L5CCK8 and (c) (C18)₂Peg2000CCK8 with the CCK8 peptide moiety able to target the cholecystokinin receptors; (d) DSPECCK8 in which CCK8 bioactive peptide is conjugated on a phospholipidic moiety.

FIG. 2 is a schematic representation of monomer molecule synthesized: (a) (C18)₂L-Lys(DTPAGlu)L2-CCK8 bearing in same molecule the hydrophobic unit having the two lipophilic tails, the chelating agent, a spacer and the bioactive peptide; (b) (C18)₂L-Lys(DTPAGlu)-PEG2000-CCK8 bearing in same molecule the hydrophobic unit having the two lipophilic tails, the chelating agent, a long PEG chain as hydrophilic spacer and the bioactive peptide.

FIG. 3 is a schematic representation of:
(a) [(C18)₂Cys-Lys(DTPAGlu)]2 in which two identical monomers of C18Cys-Lys(DTPAGlu) are covalently connected by a disulphide bond; (b) [(C18)₂CysCCK8]2 in which two identical onomers of C18CysCCK8 are covalently connected by a disulphide bond.

FIG. 4 is a schematic presentation of a rodlike micelle and a vesicle formulated to start from monomers reported in FIG. 1-2.

FIG. 5 is a Cryo-TEM image of (C18)₂DTPAGlu(C18)₂Peg2000CCK8 mixed vesicles prepared according to the preparation mode 2.

FIG. 6 shows the binding to receptor expressing and control cells at 4° C. The aggregates bind with high affinity the CCKBR expressing cells (first column from the left). They can be displaced by addition of 10 μM CCK8 peptide (second column from left). Binding to non receptor expressing cells (third column) is negligible and similar to background levels obtained with no cells in the medium (fourth column).

FIG. 7 shows a gamma camera image of the mouse. The receptor overexpressing tumour (on the left) is compared to the control tumour (on the right). The bright area in the centre of image is due to the elevated radioactivity levels in blood and in all high blood pool organs (liver and spleen).

FIG. 8 shows the cell survival (MTT assay, %) of A431 cells overexpressing CCKBR and control (receptor negative) cells as consequence of the Doxorubicin activity delivered by supramolecular aggregates.

The following Examples further illustrate the invention.

### EXAMPLE 1

### Preparation of (C18)₂DTPAGlu Monomer and its Gd(III) Complex

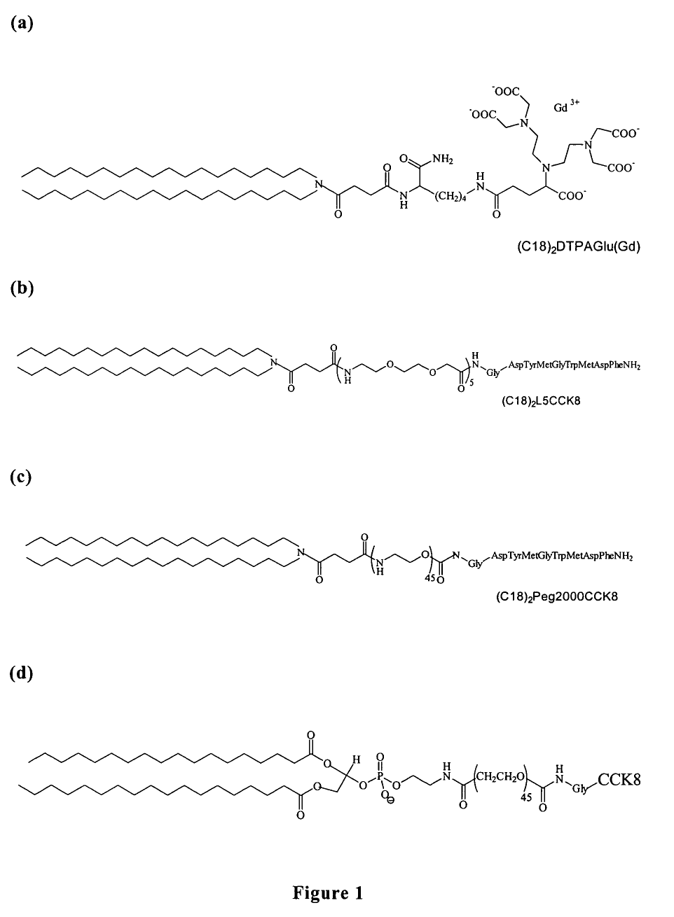

The (C18)₂DTPAGlu monomer, shown in FIG. 1(a), was synthesized by SPPS approach by using a polymeric support as disclosed in Chang, W. C. and White, P. D.; Fmoc solid phase peptide synthesis; Oxford Univ. Press (2000), New York. A Rink-amide MBHA resin (0.78 mmol/g, 0.5 mmol scale, 0.64 g) was used because it releases the peptide carboxamide at the C-terminus.

The Fmoc protecting group was removed from the resin by DMF/Pip (80/20) mixture. 1.248 g (2.0 mmol) of Fmoc-Lys(Mtt)-OH activated by 1 equivalent of PyBop and HOBt and 2 equivalents of DIPEA in DMF were coupled on the resin stirring the slurry suspension for 1 h. The solution was filtered and the resin washed with three portions of DMF and three portions of DCM. The Mtt-protecting group was removed by treatment with DCM/TIS/TFA (94:5:1) mixture. The peptide-resin was stirred with 3.0 ml of this solution for 2 min. This procedure was repeated several times until the solution became colourless. The resin was washed 3 times by DCM and 3 times by DMF. At this point, the DTPAGlu-pentaester chelating agent was linked, through its free carboxyl function, to the ε-NH₂ of the lysine residue. The DTPAGlu-pentaester, N,N-Bis[2-[bis[2-(1,1-dimethyletoxy)-2-oxoethyl]-amino]ethyl]-L-glutamic acid 1-(1,1-Dimethylethyl)ester, required in the synthesis was made according to Anelli, P. L. et al., Bioconjugate Chem. 10 (1999), 137. This coupling step was performed using 2 equivalents of DTPAGlu-pentaester and HATU and 4 equivalents of DIPEA in DMF. The coupling time, compared with the classical solid phase peptide synthesis protocol, was increased up 2 hours and the reaction was checked to see if completed by using Kaiser test. Then, the N-terminal Fmoc protecting group was removed and 0.622 g (4.0 mmol) of N,N-dioctadecylsuccinamic acid in DMF/DCM (1/1) mixture were condensed. N,N-dioctadecylsuccinamic acid was synthesized according to known method (Tampé R., J. Am. Chem. Soc., 1994, 116, 8485-8491). Coupling was repeated twice under N₂ flow for 1 h. The liphophilic moiety was activated in situ by the standard HOBt/PyBop/DIPEA procedure. For deprotection and cleavage, the fully protected fragment was treated with TFA containing TIS (2.0%) and water (2.5%). The crude product was precipitated at 0° C., and washed several times with small portions of water and recrystallized from methanol and water. The corresponding gadolinium chelates (Gd—(C18)₂DTPAGlu) has been carried out by adding light excess of the GdCl₃ to the aqueous solution of the monomeric chelating agent ((C18)₂DTPAGlu) at the neutral pH and room temperature. The formation of Gd³⁺ complex ((C 18)₂DTPAGlu), was followed by measuring the solvent proton relaxation rate (1/T₁). The purity of the imaging agents was checked by measuring the Gd content by usual means (decomplexing in 2N hydrochloric acid and titrating with EDTA solution; indicator, Xylenol-orange) and gave results substantially close to theoretical values. The product was identified by Maldi-T of mass spectrometry.

(C18)₂DTPAGlu: MW=1195 u.m.a.

### EXAMPLE 2

### Preparation of (C18)₂L5CCK8 and (C18)₂Peg2000CCK8 Monomers

(C18)₂L5CCK8 and (C18)₂Peg2000CCK8 monomers (FIG. 1(b-c) were synthesized by SPPS approach under standard conditions using Fmoc strategy.

Rink-amide MBHA resin (0.78 mmol/g, 1 mmol scale, 1.28 g) was used.

The peptide chain was elongated by sequential coupling and Fmoc deprotection of the following Fmoc-amino acid derivative: Fmoc-Phe-OH, Fmoc-Asp(OtBu)-OH, Fmoc-Met-OH, Fmoc-Trp(Boc)-OH, Fmoc-Gly-OH, Fmoc-Met-OH, Fmoc-Tyr(tBu)-OH, Fmoc-Asp(OtBu)-OH, Fmoc-Gly-OH. All couplings were performed twice for 1 hour, by using an excess of 4 equivalents for the single amino acid derivative. The α-amino acids were activated in situ by the standard HOBt/PyBop/DIPEA procedure. DMF was used as a solvent. Fmoc deprotection was carrier out by 20% solution of piperidine in DMF after the coupling of each amino acidic residue. The coupling steps were monitored by the qualitative Kaiser test. When the peptide synthesis was complete, the Fmoc N-terminal protecting group was removed and the peptide-resin was shared in 2 portions.

The first portion of H-Gly-CCK8-resin (0.50 mmol) was elongated by the coupling reaction of five residues of Fmoc-AdOO—OH. This amino acidic derivate was condensed to the α-NH₂ of the glycine residue in a single coupling by using an excess of 2 equivalents. PyBop, HOBt and DIPEA were adding to activate the condensation reactions. The reaction time required was about 60 min under N₂ flow at room temperature.

When all five linkers were coupled on the peptide chain, the N,N-dioctadecylsuccinamic acid was bound. The coupling was carried out by an excess of 4 equivalents (1.244 g, 2.0 mmol) of the lipophilic compound dissolved in 3 ml of DMF/DCM (50/50) mixture. 0.520 g (2.0 mmol) of PyBop, 0.306 g (2.0 mmol) of HOBt and 670 μl (4.0 mmol) of DIPEA, dissolved in DMF were introduced in the vessel like activating agents. The coupling time was 1 h under N₂ flow at room temperature.

Yield for aliphatic acid coupling, monitored by the Kaiser test, was in the range 95-98%. For deprotection and cleavage, the fully protected resin was treated with the TFA/TIS/EDT/H₂O (93/2/2.5/2.5) mixture, and the free product precipitated at 0° C. by adding water drop wise. The homogeneity of the crude product was checked by analytical HPLC; the crude product presented a main peak with a retention time of 26.2 minutes (column: reverse phase C18, eluents: water with 0.1% TFA (A) and acetonitrile with 0.1% TFA(B); elution gradient: from 60% to 80% B in 10 minutes and from 80% to 95% B in 15 minutes. The product was purified by preparative HPLC, with the same separation method as used on an analytical scale. The purity of the product was confirmed with Maldi mass spectroscopy, which showed the peak at the expected mass value.

(C18)₂L5CCK8: Rₜ=26.2 min; MW=2243 u.m.a.

On second portion of peptide-resin (0.50 mmol) was condensed the Fmoc-NH-Peg2000-NHS residue. The coupling was performed in DMF twice for 1 hour, by using an excess of 1.5 equivalents. The carboxy-polyethylene glycol was activated in situ by the standard HOBt/PyBop/DIPEA procedure. The coupling reaction was monitored by the qualitative Kaiser test. Fmoc deprotection was carried out by 20% solution of piperidine in DMF. Then, N,N-dioctadecylsuccinamic acid was bound on the α—NH₂ of PEG2000 hydroxylic spacer by using the same experimental conditions of the (C18)₂L5CCK8 synthesis.

The monomer-resin was treated with an acidic solution of TFA/TIS/H₂O (95.5/2.0/2.5) mixture. The crude product was precipitated at 0° C. by adding water dropwise. The homogeneity of the crude product was checked by analytical HPLC; the crude product presented a main peak with a retention time of 27.1 minutes (column: reverse phase C18, eluents: water with 0.1% TFA (A) and acetonitrile with 0.1% TFA(B); elution gradient: from 5% to 70% B in 30 minutes. The product was purified by preparative HPLC, with the same separation method as used on an analytical scale. The purity of the product was confirmed with Maldi mass spectroscopy, which showed the peak at the expected mass value.

(C18)₂PEG2000CCK8, Rₜ=27.1 min; MW=3746 u.m.a.

### EXAMPLE 3

### Formulation of Mixed Micellar Aggregates with (C18)₂DTPAGlu-(C18)₂L5CCK8

All aggregate solutions were prepared by weighting, buffering the samples at pH 7.4 with 0.10 M phosphate and 34 mM NaCl. In a first preparative mode (mode 1), the monomer's solutions were stirred at room temperature until complete dissolution and then used without further treatment. The aggregates obtained were in micellar form (labelled “M-L5”).

In (C18)₂DTPAGlu-(C18)₂L5CCK8 mixed solution, the imposed molar ratio between the solutes was 5 to 1 (chelator containing vs. peptide containing). The structural characterization of the aggregates was obtained by Small-Angle Neutron Scattering (SANS) measurements. The SANS measurements were performed at 25° C. on the KWS2 located at the Forschungszentrum Julich, Germany. Neutrons with an average wavelength of 7 Å and wavelength spread Δλ/λ<10% were used. A two-dimensional arrays detector at two different sample-to-detector distances, 2 and 8 m detected neutrons scattered from the sample. These configurations allowed collecting the scattered intensity in a range of moment transfer Q between 0.003 and 0.12 Å⁻¹. Samples were contained in 1 mm path length quartz cells and measurements times ranged between 20 min to 2 h.

The longitudinal water proton relaxation rates of micellar aggregates were measured on a Stelar Spinmaster (Mede, Pavia, Italy) spectrometer operating at 20 MHz, by means of the standard inversion-recovery technique (16 experiments, 2 scans). A typical 90° pulse width was 4 μs and the reproducibility of the T₁ data was ±0.5%. The proton 1/T₁ NMRD profiles were measured over a continuum of magnetic field strength from 0.00024 to 0.28 T (corresponding to 0.01-12 MHz proton Larmor Frequency) on a Stelar Fast Field-Cycling relaxometer. This relaxometer works under complete computer control with an absolute uncertainty in 1/T₁ of ±1%. Data points at 20 MHz and 90 MHz were added to the experimental NMRD profiles and were recorded on the Stelar Spinmaster (20 MHz) and on a JEOL EX-90 (90 MHz) (Tokyo, Japan) spectrometer, respectively.

Table 1 reports the aggregation numbers (N_{agg}), micellar charge (z), the radii and the heights of the cylindrical aggregates (R and h in Å), the fraction of chelating agent in the aggregates (f) and the relaxivity values (r₁ₚ in mM⁻¹ s⁻¹) normalized to 1 mM concentration of Gd(III) ions.

TABLE 1
Structural parameters of the pure and mixed aggregates
present in the considered micelle systems determined by Small-Angle Neutron
Scattering (SANS) and measured relaxivity values determined by NMRD
profiles at 20 MHz and 25° C.

| Systems | N_{agg} | z | R | h | n_{w} | f | r₁ₚ |
|---|---|---|---|---|---|---|---|
| (C18)₂DTPAGlu | 102 ± 5 | −60 ± 5 | 39 ± 3 | 108 ± 3 | 95 ± 8 | — | |
| (C18)₂DTPAGlu(Gd) | 88 ± 5 | −27 ± 4 | 34 ± 2 | 123 ± 4 | 94 ± 7 | — | 17.5 |
| (C18)₂DTPAGlu- | 85 ± 4 | −50 ± 4 | 34 ± 4 | 125 ± 4 | 125 ± 10 | 0.611 | |
| (C18)₂L5CCK8 | | | | | | | |
| (C18)₂DTPAGlu(Gd)- | 82 ± 4 | −20 ± 4 | 33 ± 5 | 173 ± 4 | 124 ± 10 | 0.635 | 16.3 |
| (C18)₂L5CCK8 | | | | | | | |

### EXAMPLE 4

### Formulation of Mixed Vesicular Aggregates with (C18)₂DTPAGlu-(C18)₂L5CCK8

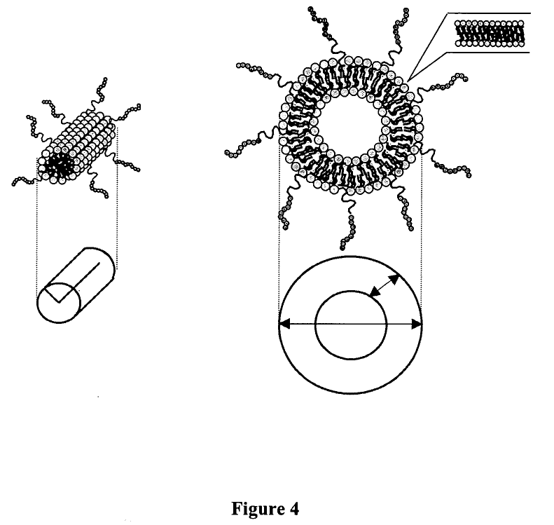

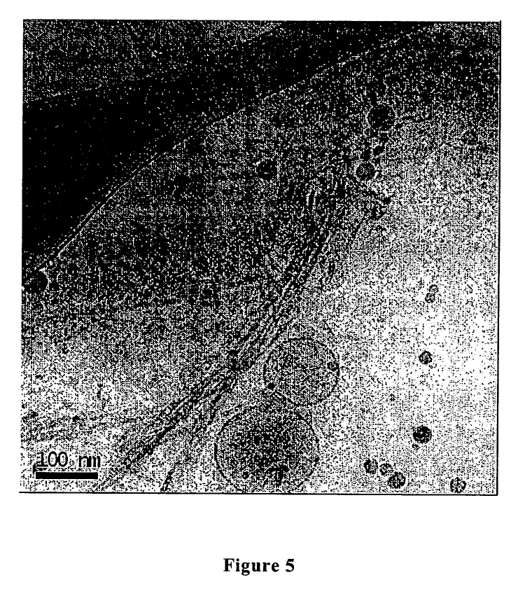

In a second preparative mode (mode 2), 10.0 mg (8.4*10⁻³ mmol) of (C18)₂DTPAGlu and 4.0 mg (1.6*10⁻³ mmol) of (C18)₂L5CCK8 were dissolved in 5 ml of a 1/1 mixture of MeOH and CHCl₃. The solution was evaporated to remove any organic solvent, and then dried in vacuum to obtain a lipid film. Moreover, the lipidic film was hydrated by vigorous vortex in 0.10 M phosphate buffer (2.0 ml) pH 7.4 for 1 hour. The mixture was further homogenised by sonication for about 15 min. Finally, vesicle size was homogenized around at 100 nm by extrusion technique. The mixture was extruded through double-stacked polycarbonate membranes (pore size from 400 to 100 nm) by using an Avanti Mini extruder from Avanti Polar Lipids (Usa, Canada). The aggregates were obtained in vesicle form (labelled “V-L5”). Their size was determined by Cryo-TEM technique (FIG. 4). The lipidic bilayer (τ=9÷13 nm) of the vesicle aggregates was calculated by SANS and Cryo-TEM techniques.

### EXAMPLE 5

### Formulation of Mixed Micellar and Vesicular Aggregates with (C18)₂DTPAGlu-(C18)₂Peg2000CCK8

The experiments of Example 3 and 4 were repeated, changing the (C18)₂L5CCK8 monomer with (C18)₂Peg2000CCK8 monomer (FIG. 1 (c)), endowed of a polyethylene glycol spacer. In the experiments, the (C18)₂DTPAGlu chelating molecule was used in complexed and uncomplexed form with the gadolinium paramagnetic ion. In a first sample, mixed aggregates were prepared according to “mode 1” to give micelle aggregates (labelled “M-Peg”) and in the second sample were prepared according to “mode 2” to give vesicles (labelled “V-Peg”).

Table 2 reports the structural parameters of pure and mixed micellar aggregates, obtained by SANS measurements and the longitudinal water proton relaxation rate obtained by NMRD profiles. In particular, the aggregation numbers (N_{agg}), micellar charge (z), the radii and the heights of the cylindrical aggregates (R and h in Å), the lipidic bilayer of the vesicles (τ in Å), the fraction of chelating agent in the aggregates (f) and the relaxivity values (r₁ₚ in mM⁻¹ s⁻¹) normalized to 1 mM concentration of Gd(III) ions are reported.

TABLE 2
Structural and relaxometric parameters of mixed micellar and
vesicular aggregates, obtained by Sans and measured relaxivity values at
20 MHz and 25° C.

| Systems | N_{agg} | z | R | h | n_{w} | τ | f | r₁ₚ |
|---|---|---|---|---|---|---|---|---|
| M-Peg | 125 ± 5 | −50 ± 4 | 32 ± 4 | 201 ± 4 | 135 ± 5 | 140 ± 5 | 0.690 | |
| M-PegGd(III) | 120 ± 5 | −20 ± 4 | 33 ± 4 | 253 ± 4 | 132 ± 4 | 140 ± 5 | 0.699 | 13.4 |

### EXAMPLE 6

### Preparation of (C18)₂L-Lys(DTPAGlu)L2-CCK8 Monomer

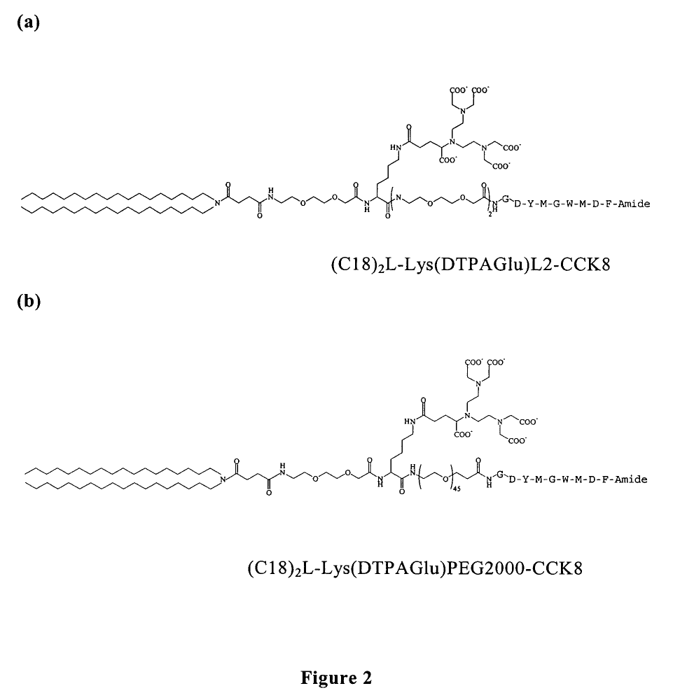

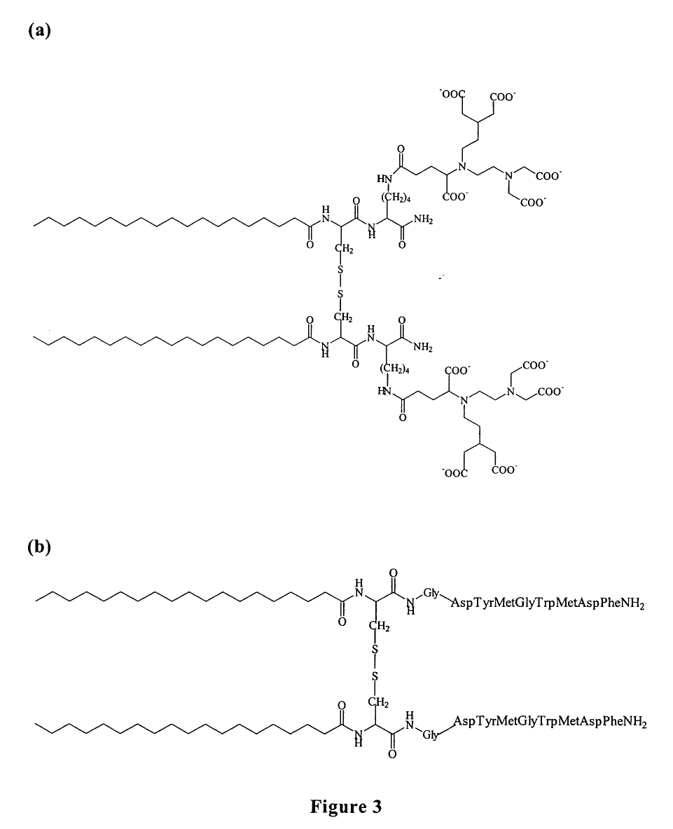

The (C18)₂L-Lys(DTPAGlu)L2-CCK8 monomer (FIG. 2.a) was synthesized by SPPS approach under standard conditions using Fmoc strategy. In the present invention the synthesis was carried out on a Rink-amide MBHA resin (0.78 mmol/g, 1 mmol scale, 1.28 g). The endogenous octapeptide CCK8 was elongated by the sequential coupling of amino acid as reported in example 2. When the peptide synthesis was complete, the Fmoc N-terminal protecting group on the last residue was removed and two Fmoc-AdOO—OH spacers were condensed in a single coupling by using an excess of 2 equivalents. The reaction time was about 60 min under N₂ flow at room temperature.

The Fmoc protecting group was removed from the spacer by DMF/Pip (80/20) mixture. 1.248 g (2.0 mmol) of Fmoc-Lys(Mtt)-OH activated by 1 equivalent of PyBop and HOBt and 2 equivalents of DIPEA in DMF were coupled on the resin stirring the slurry suspension for 1 h. The solution was filtered and the resin washed with three portions of DMF and three portions of DCM. The Mtt-protecting group was removed by treatment with DCM/TIS/TFA (94:5:1) mixture. The peptide-resin was stirred with 3.0 ml of this solution for 2 min. This procedure was repeated several times until the solution became colorless. The resin was washed 3 times by DCM and 3 times by DMF. The DTPAGlu-pentaester chelating agent was linked to the ε-NH₂ of the lysine residue, as described in example 1. Another Fmoc-AdOO—OH linker was condensed on the principal chain of the lysine residue. Then the N-terminal Fmoc protecting group was removed and 2.488 g (4.0 mmol) of N,N-dioctadecylsuccinamic acid in DMF/DCM (1/1) mixture were condensed. Coupling was repeated twice under N₂ flow for 1 h. The liphophilic moiety was activated in situ by the standard HOBt/PyBop/DIPEA procedure. For deprotection and cleavage, the fully protected fragment was treated with TFA containing TIS (2.0%) and water (2.5%).

The homogeneity of the crude product was checked by analytical HPLC; the crude product presented a main peak with a retention time of 26.2 minutes (column: reverse phase C4, eluents: water with 0.1% TFA (A) and acetonitrile with 0.1% TFA(B); elution gradient: from 20% to 95% B in 25 minutes. The product was purified by preparative HPLC, with the same separation method as used on an analytical scale. The purity of the product was confirmed with Maldi mass spectroscopy, which showed the peak at the expected mass value.

(C18)₂L-Lys(DTPAGlu)L2-CCK8; Rₜ=29.5 min; MW=2733 u.m.a.

In this experiment the (C18)₂L-Lys(DTPAGlu)L2-CCK8 monomer labelled as “(C18)₂-Y” (FIG. 1(d)) and its corresponding gadolinium chelate (Gd—(C18)₂—Y) were used according to the mode 1 preparation. The aggregate solution was prepared dissolving 27.3 mg of lyophilized sample in 2.0 ml of 0.10 M phosphate buffer at pH 7.4. and 34 mM NaCl.

### EXAMPLE 7

### Preparation of (C18)₂L-Lys(DTPAGlu)-PEG2000-CCK8 Monomer

The (C18)₂L-Lys(DTPAGlu)-PEG2000-CCK8 monomer (FIG. 2.b) was synthesized by SPPS approach under standard conditions using Fmoc strategy. In the present invention the synthesis was carried out on a Rink-amide MBHA resin (0.78 mmol/g, 1 mmol scale, 1.28 g). This monomer, as well as the monomer described in Example 6, contains contemporary both the bio-specific peptide and the chelating agent. The endogenous octapeptide CCK8 was elongated by the sequential coupling of amino acid as reported in the example 2. When the peptide synthesis was complete, the Fmoc N-terminal protecting group on the last residue was removed and the Fmoc-NH-Peg2000-NHS residue was coupled performed in DMF twice for 2 hour, by using an excess of 1.5 equivalents. The carboxy-polyethylene glycol was activated in situ by the standard HOBt/PyBop/DIPEA procedure. The coupling reaction was monitored by the qualitative Kaiser test. Fmoc deprotection was carrier out by 20% solution of piperidine in DMF. 1.248 g (2.0 mmol) of Fmoc-Lys(Mtt)-OH activated by 1 equivalent of PyBop and HOBt and 2 equivalents of DIPEA in DMF were coupled on the resin stirring the slurry suspension for 1 h. The solution was filtered and the resin washed with three portions of DMF and three portions of DCM. The Mtt-protecting group was removed by treatment with DCM/TIS/TFA (94:5:1) mixture. The peptide-resin was stirred with 3.0 ml of this solution for 2 min. This procedure was repeated several times until the solution became colorless. The resin was washed 3 times by DCM and 3 times by DMF. The DTPAGlu-pentaester chelating agent was linked to the ε-NH₂ of the lysine residue, as described in example 1. Another Fmoc-AdOO—OH linker was condensed on the principal chain of the lysine residue. Then the N-terminal Fmoc protecting group was removed and 2.488 g (4.0 mmol) of N,N-dioctadecylsuccinamic acid in DMF/DCM (1/1) mixture were condensed. Coupling was repeated twice under N₂ flow for 1 h. The liphophilic moiety was activated in situ by the standard HOBt/PyBop/DIPEA procedure. For deprotection and cleavage, the fully protected fragment was treated with TFA containing TIS (2.0%) and water (2.5%).

The homogeneity of the crude product was checked by analytical HPLC; the crude product presented a main peak with a retention time of 26.2 minutes (column: reverse phase C4, eluents: water with 0.1% TFA (A) and acetonitrile with 0.1% TFA(B); elution gradient: from 20% to 95% B in 25 minutes. The product was purified by preparative HPLC, with the same separation method as used on an analytical scale. The purity of the product was confirmed with Maldi mass spectroscopy, which showed the peak at the expected mass value.

(C18)₂L-Lys(DTPAGlu)-PEG2000-CCK8; Rₜ=26.5 min; MW=4462 u.m.a.

In this experiment the (C18)₂L-Lys(DTPAGlu)-PEG2000-CCK8 monomer labelled as “(C18)₂-Y-PEG2000” (FIG. 2.b)) and its corresponding gadolinium chelate (Gd—(C18)₂-Y-PEG2000) were used according to the mode 2 preparation. The aggregate solution was prepared dissolving 4.5 mg of lyophilized sample in 2.0 ml of 0.10 M phosphate buffer at pH 7.4 and 34 mM NaCl. The mixture was further homogenised by sonication for about 30 min. Finally, vesicle size was homogenized around at 100 nm by extrusion technique.

### EXAMPLE 8

### DOPC/(C18)₂-Y-PEG2000 Liposome Preparation and Doxorubicin Loading

The liposomes were composed of DOPC/(C18)₂-Y-PEG2000 at different molar ratios (90:10, 80:20, 70:30, 60:40, 50:50). Briefly, lipids were dissolved in a chloroform/methanol (50:50) mixture and evaporated to remove any organic solvent, and then dried in a vacuum to obtained a lipidic film. The lipidic film was hydrated by vortex in a 0.1 M citrate-phosphate buffer at pH 4.0. The mixture was sonicated for 30 min at 60° C. and extruded through double-stacked polycarbonate membranes (pore sizes from 400 to 100 nm) using an extruding device from Avanti Polar lipids (Alabaster, Ala.). Then, pH of liposome solution was brought at 7.0 by adding small aliquots of 1 M NaOH solution. Final concentration of liposomes was 7·10⁻⁴ M. 3.5·10⁻⁴ M stock solution of Doxorubicin (DOXO) was prepared by weighting 1.0 mg of DOXO in 5.0 mL of 5 mM phosphate buffer pH 7.4 and 0.9% wt NaCl. Loading of DOXO in liposome was performed under pH gradient by mixing liposomes with a DOXO solution. The DOXO content in the liposomes reaches 0.2/1.0 drug/lipid molar ratio. Free DOXO was then removed by passing through a gel filtration column in the buffer of HEPES 25 mM and NaCl 150 mM (pH 7.2).

### EXAMPLE 9

### Synthesis of DSPECCK8 Monomer and (C18)₂DTPAGlu-DSPECCK8 Mixed Liposomes Formulation

The DSPECCK8 monomer (FIG. 1.d) was synthesized by SPPS approach under standard conditions using Fmoc strategy.

Rink-amide MBHA resin (0.78 mmol/g, 1 mmol scale, 1.28 g) was used.

The peptide chain was elongated by sequential coupling and Fmoc deprotection of the amino acid residues according to example 2. When the peptidic synthesis was complete and the Fmoc N-terminal protecting group removed DSPE phospholypid was condensed on the α-NH₂ of glycine residue. The coupling was performed in DMF/NMP/DCM mixture twice for 4 hours, by using an excess of 2 equivalents. The coupling reaction was monitored by the qualitative Kaiser test. The monomer-resin was treated with an acidic solution of TFA/TIS/H₂O (95.5/2.5/2.5) mixture. The crude product was precipitated at 0° C. by adding ether dropwise. The crude product was checked by analytical thin layer chromatography, and phosphorus determination. The product was purified by dialysis procedure. The purity of the product was confirmed with Maldi mass spectroscopy, which showed the peak at the expected mass value.

DSPECCK8, MW=3950.5 u.m.a.

(C18)₂DTPAGlu-DSPECCK8 mixed liposomes were prepared according to “mode 2” to give liposomes. 6.0 mg (1*10⁻² mmol) of (C18)₂DTPAGlu and 4.0 mg (3.3*10⁻³ mmol) of DSPECCK8 were dissolved in 5 ml of a 1/1 mixture of MeOH and CHCl₃. The solution was evaporated to remove any organic solvent, and then dried in vacuum to obtain a lipid film. Moreover, the lipidic film was hydrated by vigorous vortex in 0.10 M phosphate buffer (5.0 ml) pH 7.4 for 1 hour. The mixture was further homogenised by sonication for about 30 min. Finally, vesicle size was homogenized around at 400 nm by extrusion technique.

### EXAMPLE 10

### Specific Binding to Cultured Cells Overexpressing a Target Receptor

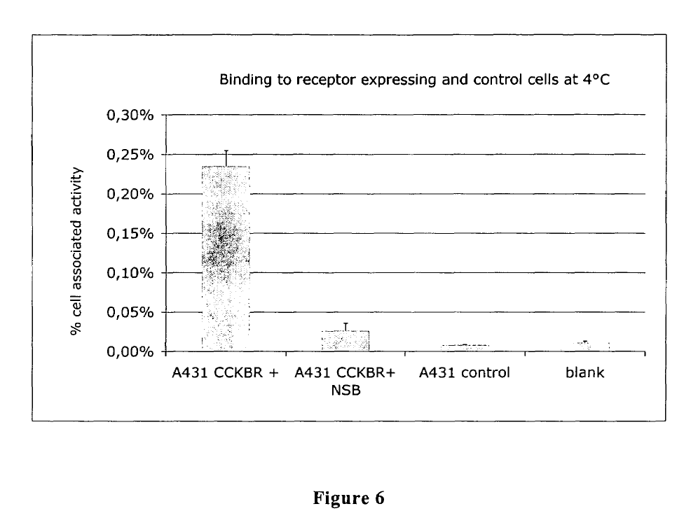

(C18)₂DTPAGlu-(C18)₂Peg2000CCK8 vesicular aggregates were prepared by dissolving monomers at a 5 to 1 ratio (chelator containing vs peptide containing) with final concentration in the 10⁻² M range. Trace amounts of ¹¹¹In Citrate were added and the suspension was allowed to incubate at room temperature for 30 min. Proof of vesicular aggregate formation and labelling with ¹¹¹In was obtained by gel filtration chromatography in comparison to In-Citrate which was run as control. The obtained vesicular aggregates were then diluted into cell culture medium containing cell suspensions with final concentration of monomer which were kept always above 10⁻³ M. After incubation for 60 min the cell bound radioactivity was separated from radioactivity in the medium by centrifugation through dibutyl phthalate. FIG. 6 shows typical results from an experiment performed at 4° C. where high binding of the aggregates to the CCKBR expressing cells can be seen (first column from the left) which can be displaced by addition of 10 μM CCK8 peptide (second column from left). Binding to non receptor expressing cells (third column) is negligible and similar to background levels obtained with no cells in the medium (fourth column). Similar results, with slightly higher absolute uptake values, were obtained when experiments were performed at 37° C.

### EXAMPLE 11

### In Vivo Specific Receptor Targeting of the Vesicular Aggregates

Vesicular aggregates prepared as described in the previous example were injected into nude mice bearing subcutaneous xenografts in opposite thighs (receptor positive in left thigh, receptor negative in right thigh). One hundred μL of ¹¹¹In labelled vesicular aggregate suspension was injected in the tail vein of the animals. After 16 h the animals were killed, imaged with a medium energy collimator equipped clinical gamma camera and subsequently dissected to evaluate organ distribution of radioactivity. The receptor overexpressing tumour clearly shows higher uptake compared to the control tumour. Radioactivity levels were elevated in blood and in all high blood pool organs (liver, spleen). There appeared to be no overall loss of radioactivity from the animals over the 16 h observation period indicating the vesicular aggregates were extremely stable. The receptor expressing tumours were the highest concentrating tissue after liver and spleen. On average CCKBR overexpressing xenografts contained 6.9±1.7% of injected dose per gram (% ID/g) against 5.1±1.1% ID/g for control tumours (mean ±SD).

### EXAMPLE 12

### Specific Doxorubicin Activity to Cultured Cells

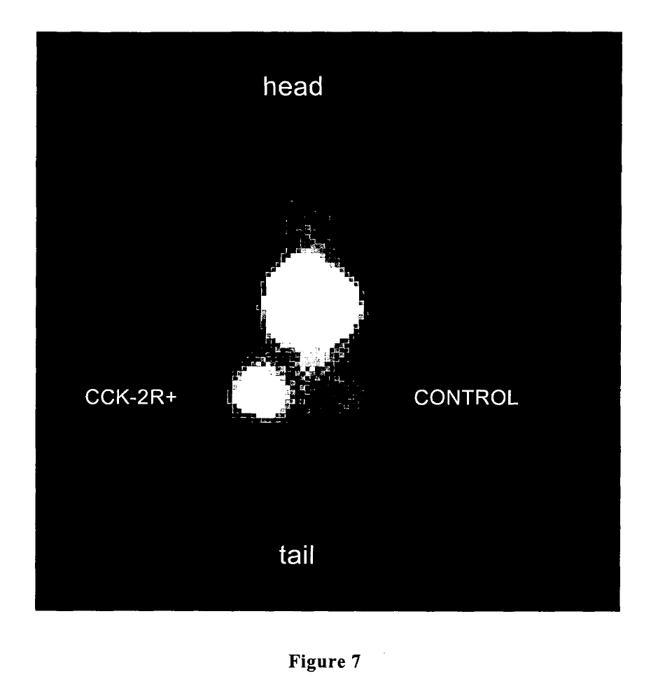

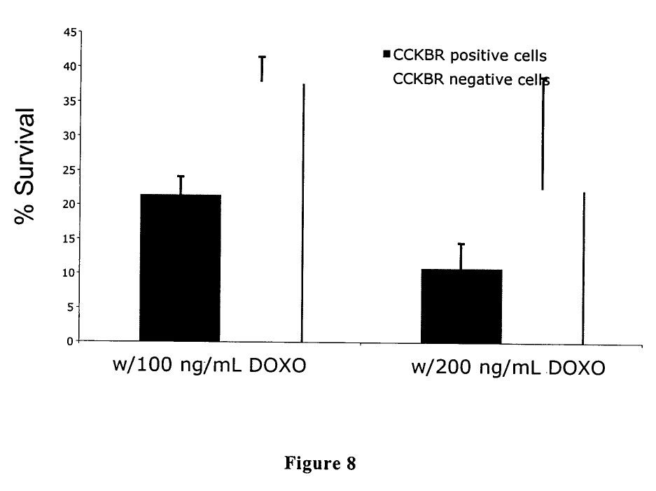

A431 cells overexpressing CCKBR and control (receptor negative) cells were incubated with doxorubicin containing aggregates, related to this invention and prepared as described in Example 8, at the given overall concentration. Cells were incubated for 96 h at which time overall survival was determined relative to control (untreated) cells using the MTT assay. Receptor overexpressing cells appear markedly more sensitive to treatment than receptor negative cells (FIG. 7).

1. A supramolecular aggregate comprising two monomers:
a first monomer containing a paramagnetic or radioactive metal ion coupled to a chelating agent having a lipophilic moiety,
a second monomer containing a bioactive peptide linked to a lipophilic moiety through an organic spacer.

2. The aggregate of claim 1 in form micelles with particle size between 5 and 50 nm, vesicles or liposomes having a size ranging between 50 and 500 nm; lipidic bilayers, double strand aggregates, having a thickness ranging between 5-20 nm.

3. The aggregate of claim 1 consisting of a single monomer bearing in the same molecule the hydrophobic unit having two lipophilic tails, the chelating agent, a spacer and the bioactive peptide.

4. The aggregate of claim 1 consisting of “Geminal” compounds in which two identical monomers are connected by a spacer group, the two monomers being possibly linked together by an amide bond, an ester or thioester bond or a disulphide bond, the spacer between the two monomers being symmetric or asymmetric and selected from an alkyl chain or an aromatic moiety.

5. The aggregate of claim 1 consisting of two monomers, containing only one lipophilic substituent on the organic moiety, linked together by a covalent bond obtained by UV-induced reaction on C—C unsatured bonds of the alkyl chain.

6. The aggregate of claim 1 further containing non-ionic, ionic and mixtures of ionic and non-ionic surfactants.

7. The aggregate of claim 1 further containing amphipatic compounds.

8. The aggregate of claim 1, wherein the bioactive branched or linear peptide have the following sequence of general formula (I) or (III)
[AA₀]_{w}-AA₁-AA₂-AA₃-Gly-Trp-AA₆-Asp-PheR₂ (I) 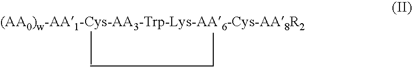
AA₀ AdOO or other spacer
AA₁ Asp or Glu
AA₂Tyr or SO₃H-Tyr
AA₃Met or Nle or Leu
AA₆ Met, Nle or Leu
AA′₁, AA′₃, AA′₆, and AA′8 are any amino acid, either natural or not, in L or D configuration; or
AA′₈, is an amino alcohol derivative from any amino acid, either natural or not, in L or D configuration.

9. The aggregate of claim 1, wherein the bioactive branched or linear peptide are peptide sequences corresponding to RGD-peptides, somatostatin, cortistatin, octreotide, bombesin, chemotactic peptides, vasoactive intestinal peptide, Her2 ligands, NPY analogs, CXCR4 ligands such as the T140 peptide, Integrin binding ligands, fibroblast growth factor, hepatocyte growth factor, epidermal growth factor, laminin binding ligands, nerve growth factor, fibronectin, fibroblast growth factor, insulin like growth factor, vascular endothelial growth factor receptor ligands, a platelet derived growth factor receptor ligands and transforming growth factor receptor ligands and mimetics thereof or ligands for other plasma membrane receptor or other cell surface component of cancer cells.

10. The aggregate of claim 1, wherein the chelating group are selected from EDTA, DTPA, DTPAGlu, DTPALys, DTPASer, BOPTA, DOTA, DO3A and/or their derivatives, containing a free function unit for covalent linkage to the other monomer units.

11. The aggregate of claim 1, wherein the paramagnetic metal ion is selected from Gd(III), Mn(II), Cr(III), Cu(II), Fe(III), Pr(III), Nd(III), Sm(III), Tb(III), Yt(III) Dy(III), Ho(III) and Er(III).

12. The aggregate of claim 1 wherein the radioactive metal is selected from ¹¹¹In(III), ^{99m}Tc(V), ⁹⁰Y(III) ¹⁷⁷Lu(III) ⁶⁸Ga(III), ⁶⁷Ga(III).

13. The aggregate of claim 1, wherein the number ratio of bioactive peptide moiety and the chelating agents is comprised between 0.5 and 10, preferably between 2 and 5.

14. A method for the preparation of the aggregates of claim 1 in form of micelles comprising:
A. the simultaneous dispersion of the two components in the aqueous carrier liquid at final concentration higher that the relative critical micellar concentration;
B. the optional addition of the third component, whereby the addition of said third component cause the dispersion to become into micellar form.

15. A method for the preparation of the aggregates of claim 1 in form of mixed vesicle or liposomes or double strands comprising:
A. Dissolving the monomer combination in a suitable organic solvent;
B. Evaporation of solvents under vacuum to give a lipid film;
C. Hydration of the lipid film in an aqueous solution by vortexing;
D. Dispersing the mixture in water or other physiologically liquid carrier;
E. Extruding the aqueous supramolecular aggregates.

16. The aggregates of claim 1 for use in RMI imaging for diagnosis, NM diagnosis and therapy.

17. Pharmaceutical or diagnostic compositions comprising the aggregates of claim 1 and a suitable excipient.

18. Pharmaceutical compositions according to claim 17 wherein the aggregates is loaded with antineoplastic agents.

19. Pharmaceutical compositions according to claim 18 wherein the antineoplastic agent is selected from doxorubicin, daunorubicin, epirubicin, esorubicin, and idarubicin.
